(19) United States
(12) Patent Application Publication
(10) Pub. No.: US 2007/0240717 A1
(43) Pub. Date: Oct. 18, 2007

(54) SYSTEM AND METHOD FOR SETTING POSITIVE END EXPIRATORY PRESSURE DURING MECHANICAL VENTILATION BASED ON DYNAMIC LUNG FUNCTION

(76) Inventors: David W. Kaczka, Baltimore, MD (US); Carissa L. Bellardine Black, St. Louis Park, MN (US); Kenneth R. Lutchen, Newton, MA (US)

Correspondence Address: GAUTHIER & CONNORS, LLP, 225 FRANKLIN STREET, SUITE 2300, BOSTON, MA, 02110, US

(21) Appl. No.: 11/695,221

(22) Filed: Apr. 2, 2007

Related U.S. Application Data
(63) Continuation of application No. PCT/US05/37631, filed on Oct. 14, 2005.
(60) Provisional application No. 60/618,823, filed on Oct. 14, 2004.

(51) Int. Cl. A61M 16/00

(52) U.S. Cl. 128/204.21

(57) ABSTRACT

A method is disclosed for determining a peak end expiratory pressure in a mechanical ventilator system for providing respiratory assistance to a patient. The method includes the steps of determining characteristic values for a plurality of frequencies at each of a plurality of peak end expiratory pressures, and selecting an optimal peak end expiratory pressure value responsive to the characteristic values.

### PRIORITY

This application claims priority to U.S. Provisional Patent Application Ser. No. 60/618,823 filed Oct. 14, 2004.

### GOVERNMENT RIGHTS

This invention was developed, in part, under Contract No. BES-0076818 from the National Science Foundation. The U.S. Government has certain rights to this invention.

### BACKGROUND

The invention generally relates to ventilation systems for maintaining patients on artificial mechanical ventilation, and relates in particular to systems for providing ventilation to patients suffering from acute respiratory distress syndrome and acute lung injury.

Current mechanical ventilators are operable in active or passive modes. With active modes, an effort by the patient triggers the delivery of a breath. With passive modes, only the ventilator is active and it delivers the breath at a pre-set breathing rate (frequency), volume per breath (tidal volume (VT)) and waveform. The most common active mode is referred to as volume support. With volume support, a pre-set flow wave shape (e.g., sinusoidal, step or ramp) is delivered via a ventilator pressure source during inspiration. Ventilator controlled solenoid valves then enable the patient to passively expire to the atmosphere. The primary goal of all waveforms is to maintain blood gas levels (O₂ and CO₂) to sustain normoxia and nomocapnia. Because ventilation via pressure produced at the mouth is not natural, another requirement is that the ventilator not produce excessive pressure at the airway opening. This can lead to barotrauma (intralung airway damage), which in turn can cause respiratory distress.

It is generally desirable to estimate the degree of airway obstruction or constriction, the distribution of constriction in the airways, and the relative stiffness and viscosity of the lung tissues. Although the mechanical status of a patient's lungs may be inferred from estimates of total respiratory or pulmonary resistance (R) and elastance (E) at a given frequency, tidal volume, or mean airway pressure, such estimates do not permit inference on the level or distribution of obstructions in the airways, or the relative stiffness or viscance of the lung or respiratory tissue. The resistance and elastance will be frequency dependent, however, in a heterogeneous respiratory system, and the frequency dependence of R and E may be derived from input impedance (Z), the complex ratio of pressure to flow during external forcing as a function of frequency.

U.S. Pat. No. 6,435,182 discloses systems and methods for providing a complex ventilator waveform (referred to therein as an Enhanced Ventilator Waveform) that includes a plurality of frequency components, for example, between about 0.1 Hz and about 8.0 Hz. The assessment of a heterogeneous system may be made by examining the resistance R and elastance E at several frequencies surrounding normal breathing rates. The behavior of the R and E spectra over this frequency range are very distinct for particular forms and degrees of lung disease. Such information is helpful in a) determining the severity of any lung disease that is present and its response to therapy and the mechanical ventilation itself; b) determining the pressures necessary at the airway opening to deliver a desired volume; and c) determining the likelihood of success in weaning the patient from the ventilator.

The determination the optimal pressure that is necessary at the airway opening, or positive end expiratory pressure (PEEP) however, is sometimes difficult, particularly for patients suffering from acute respiratory distress syndrome and acute lung injury. Acute respiratory distress syndrome (ARDS) and acute lung injury (ALI) are characterized by heterogeneous regional alveolar flooding and/or collapse of lung tissue. This results in hypoxemic respiratory failure necessitating the use of mechanical ventilation to sustain life. Due to the heterogeneous distribution of the disease, ventilation and gas exchange are severely compromised, and the extremes of mechanical stress imposed by controlled ventilation may propagate and exacerbate the lung injury.

The pressure (P) may be determined by the relationship P=RQ+EΔV where Q is air flow at the airway opening and ΔV is the change in volume. A pressure versus volume (P-V) graph may then be developed for a particular frequency, and the PEEP may then be set to the lower knee of a P-V curve as shown at 10 in FIG. 1, or the midpoint 12 between the lower knee 10 and the upper knee 14. For example, U.S. Pat. No. 6,907,881 discloses systems and methods for varying the peak inspiratory pressure in a mechanical ventilation system. The peak inspiratory pressure is disclosed to deviate about a mean that is chosen to correspond with a knee in a P-V curve of the lung.

In some respiratory systems, however, there may be no clear lower knee or even upper knee, as the P-V graph may be, for example, fairly linear throughout the full range. Moreover, because the P-V graph is dependent on frequency, it is difficult to know the optimal PEEP value for any particular heterogeneous system. For example, FIG. 2A shows computerized tomography (CT) scans of a healthy lung at end expiratory and full lung recruitment, as well as P-V graphs for normalized volume portions and for total volume. The end expiratory lung is shown at 20, and the full recruitment lung is shown at 22. The P-V graphs for the normalized volumes of the upper, middle and lower regions are shown at 24, 26 and 28, and are fairly similar to one another. The P-V graph for the total volume is shown at 30.

FIG. 2B shows CT scans of an unhealthy lung at end expiratory and full lung recruitment, as well as P-V graphs for the normalized partial and total volumes. The end expiratory lung is shown at 32, and the full recruitment lung is shown at 34. The P-V graphs for the normalized volumes of the upper, middle and lower regions are shown at 36, 38 and 40. The P-V graph for the total volume is shown at 42. Note that while the PV graphs 36, 38 and 40 for the normalized volumes clearly show that the lung is unhealthy, this information is not apparent when viewing only the P-V graph 42, which appears very similar to the P-V graph 30 as shown in FIG. 2A.

The optimal way to set positive end expiratory pressure and tidal volume (V_{T}) remains uncertain notwithstanding the goals of maximizing lung recruitment while minimizing overdistension of the lung. As discussed above, one method employs the contours of the inspiratory static pressure volume curve to set the upper and lower pressure bounds for mechanical ventilation. The shape of the static P-V curve lends little insight however, into the distribution of disease or the degree of recruitment during continuous ventilation. No single point or feature of the P-V curve, therefore, will provide an optimal setting in certain heterogeneous conditions. Another approach to assess regional aeration in the lung is to employ computed tomography. Computed tomography (CT) has emerged as a useful tool to assess heterogeneity of airway and parenchymal disease, and is the current gold standard for assessing the impact of PEEP on the distribution of aeration in ARDS. The use of CT clinically at the bedside, however, is currently impractical. Furthermore, the static distribution of aeration determined by CT scans at a given PEEP may not be sufficient for predicting how mechanical ventilation impacts lung function, because mechanical ventilation is a dynamic, cyclic process.

There remains a need, therefore, for more efficient and effective methods for guiding the optimal selection of PEEP in individual patients.

### SUMMARY

The invention provides a method for determining a peak end expiratory pressure in a mechanical ventilator system for providing respiratory assistance to a patient. In accordance with an embodiment, the method includes the steps of determining characteristic values for a plurality of frequencies at each of a plurality of peak end expiratory pressures, and selecting an optimal peak end expiratory pressure value responsive to the characteristic values.

In accordance with another embodiment, the invention provides a method that includes the steps of determining characteristic values for a plurality of frequencies at each of a plurality of peak end expiratory pressures, identifying an identified peak end expiratory pressure value for which the characteristic values are most linear for the plurality of frequencies, and selecting an optimal peak end expiratory pressure value responsive to the identified peak end expiratory pressure value.

In accordance with another embodiment, the invention provides a system a system for determining a peak end expiratory pressure in a mechanical ventilator system for providing respiratory assistance to a patient. The system includes collection means, identification means, and selection means. The collection means is for determining characteristic values for a plurality of frequencies at each of a plurality of peak end expiratory pressures. The identification means is for identifying an identified peak end expiratory pressure value for which the characteristic values are most linear for the plurality of frequencies. The selection means is for selecting an optimal peak end expiratory pressure value responsive to the identified peak end expiratory pressure value.

### BRIEF DESCRIPTION OF THE DRAWINGS

The following description may be further understood with reference to the accompanying drawings in which:

FIG. 1 shows an illustrative diagrammatic view of a pressure versus volume curve of a ventilator system of the prior art;

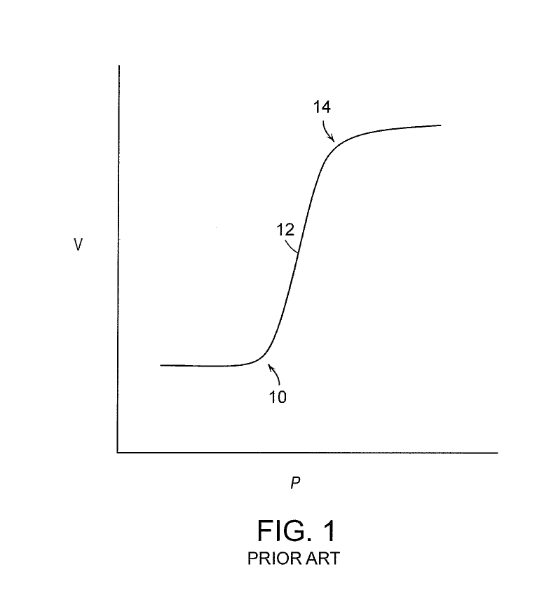

FIG. 2A shows illustrative diagrammatic CT scans and pressure versus volume curves of a healthy lung in a ventilator system of the prior art;

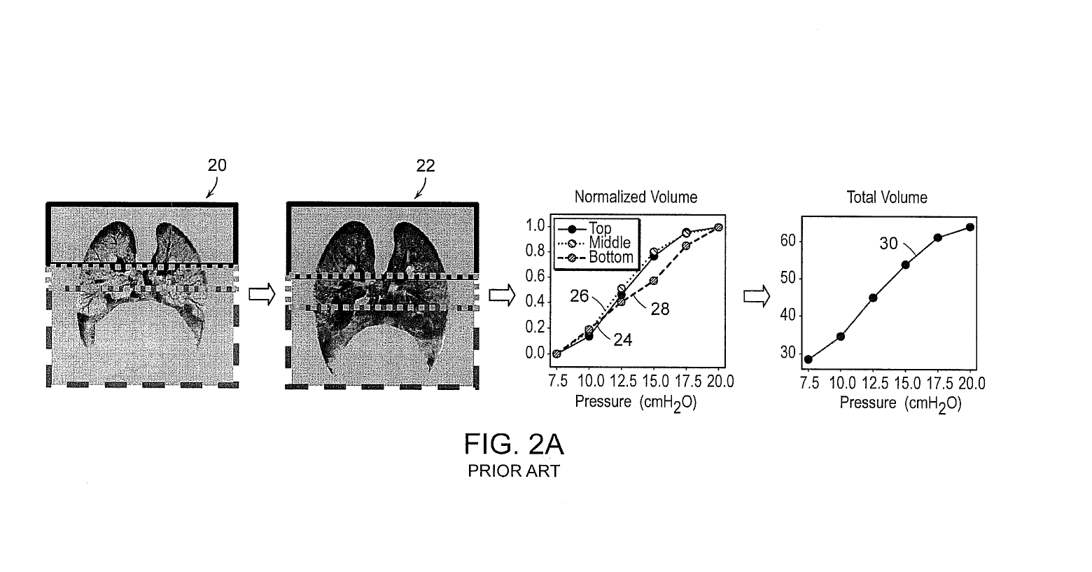

FIG. 2B shows illustrative diagrammatic CT scans and pressure versus volume curves of a an unhealthy lung in a ventilator system of the prior art;

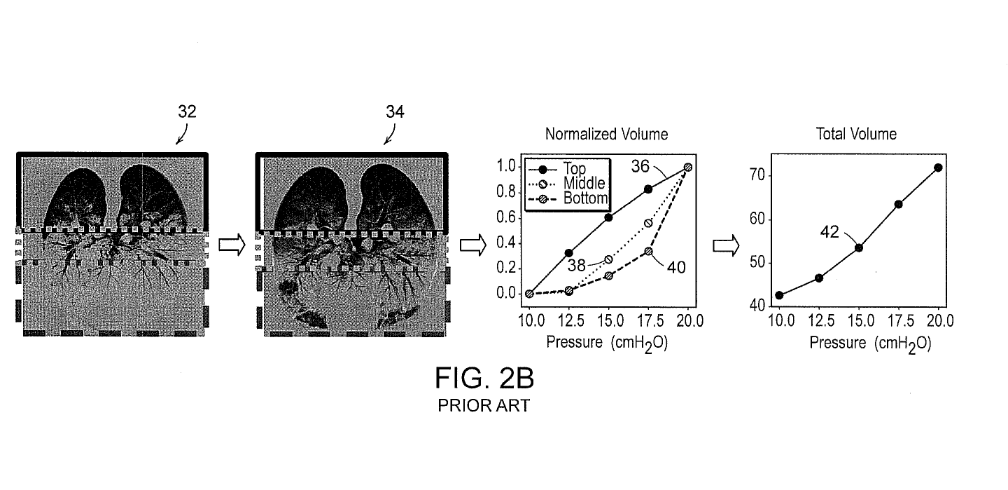

FIG. 3 shows illustrative diagrammatic tissue volume per PEEP level for a system in accordance with an embodiment of the invention;

FIG. 4 shows a table of PEEP dependence of gas exchange and hemodynamic parameters in a system in accordance with an embodiment of the invention;

FIG. 5 shows an illustrative diagrammatic representation total volume for predefined aeration compartments as a function PEEP in a system in accordance with an embodiment of the invention;

FIG. 6 shows an illustrative diagrammatic representation of CT scans taken in at various levels of PEEP in a system in accordance with an embodiment of the invention;

FIGS. 7A-7C show illustrative diagrammatic representations of Pa_{O2}, Pa_{CO2} and peak-to-peak ventilation pressures as a function of PEEP in a system in accordance with an embodiment of the invention;

FIGS. 8A and 8B show illustrative diagrammatic representations of sample dynamic resistance and elastance spectra as a function of PEEP in a system in accordance with an embodiment of the invention; and

FIGS. 9A-9C show illustrative diagrammatic representations of change in R across the range of frequencies 0.2 Hz to 8.0 Hz (Rₕₑₜ), E at 0.2 Hz (E_{low}), and static elastance (Eₛₜₐₜ) as a function of PEEP in a system in accordance with an embodiment of the invention.

The drawings are shown for illustrative purposes.

### DETAILED DESCRIPTION OF THE ILLUSTRATED EMBODIMENTS

It has been discovered that the frequency dependence of lung resistance and elastance reflect mechanical heterogeneities present in the lung, and that knowledge of how PEEP affects the heterogeneity of lung function should reflect the trade-off between recruitment and overdistension during dynamic ventilation.

In accordance with various embodiments, the invention provides that the frequency dependence of dynamic lung mechanics may be used in selecting PEEP. Specifically, it is hypothesized that an optimal PEEP may be identified that minimizes the frequency dependence of R and E corresponding to minimizing heterogeneity, and consequently maximizing recruitment and avoiding significant overdistension, while providing sufficient gas exchange, and reducing ventilation pressures.

Acute respiratory distress syndrome and acute lung injury are characterized by heterogeneous flooding and/or collapse of lung tissue. An important consideration in managing these diseases is to set mechanical ventilation so as to minimize the impact of disease heterogeneity on lung mechanical stress and ventilation distribution. It has been discovered that changes in lung mechanical heterogeneity with increasing positive end expiratory pressure in a subject model of acute lung injury may be detected from the frequency response of resistance and elastance. In accordance with an example described below, a saline lavage-induced model of lung injury in an animal was employed, and the impact of PEEP on recruitment and overdistension as assessed by CT, gas exchange, pulmonary hemodynamics, as well as static and dynamic lung mechanical characteristics were examined.

Whole-lung CT scans were acquired in five saline-lavaged subjects together with oxygenation, static elastance, and dynamic respiratory resistance and elastance at end-expiratory pressure levels of 7.5-20 mH₂O. As end-expiratory pressure increased, CT determined alveolar recruitment significantly increased from 15-20 cmH₂O but was accompanied by significant alveolar overdistension at 20 cmH₂O. An optimal range of end-expiratoly pressures (15-17.5 cmH₂O) was discovered where alveolar recruitment was maximized without significant overdistension. It has further been discovered that this range corresponded to the end-expiratory pressure levels that maximized oxygenation, minimized peak-to-peak ventilation pressures, and minimized indices reflective of the mechanical heterogeneity (e.g., frequency dependence of respiratory resistance and elastance). Static elastance did not show any significant pressure dependence or reveal an optimal end-expiratory pressure level. Dynamic mechanics, therefore, may be used to analyze lung mechanical heterogeneity and maximize oxygenation in this lung injury model. By monitoring dynamic respiratory resistance and elastance, ventilator settings may be tuned to optimize lung function.

The study was approved by the Institutional Animal Care and Use Committee at Tufts University Cummings School of Veterinary Medicine. The subjects were used in the study were 5 female sheep having a mean weight of 48 kg±2 kg. All measurements were performed while the subject was in the prone position under general anesthesia, induced with 7 mg/kg of intravenous ketamine and 0.25 mg/kg of diazepam. Anesthesia was maintained with continuous intravenous drip of pentothal. A standard 10-mm ID endotracheal tube was inserted orotracheally under fiberoptic guidance and secured in place.

Mechanical ventilation was provided by a commercially available ventilator (the NPB840 ventilator product sold by Puritan Bennett/Tyco Healthcare of Pleasanton, Calif.). First, the subject was stabilized with tital volume (V_{T})=10 ml/kg, frequency (f)=16 breaths/min, positive end expiratory pressure or PEEP=5 cmH₂O, and inspiratory to expiratory ratio of I:E=1:3, and fraction of inspired oxygen of fraction of inspired O₂ (F_{I}O₂)=1. Tidal volume (V_{T}) was adjusted to maintain a stable baseline end-tidal arterial partial pressure of CO₂ (Pa_{CO2}) between 35 and 45 mmHg.

Following induction of general anesthesia and line placement, lung injury was induced by repetitive whole-lung saline lavage. Briefly, the subject was disconnected from the ventilation circuit and warm 0.15M NaCl (40 ml/kg bwt) was instilled by gravity into the lung via endotracheal tube. After 45s, the saline was passively drained and the endotracheal tube and trachea suctioned. This procedure was repeated every 10 m until the arterial partial pressure of O₂ (PₐO₂) fell below 90 mmHg, and then the subjects were ventilated for one hour following the last lavage to assure stability of the model (i.e., hypoxemia, increased lung elastance).

Measures of gas exchange, hemodynamic, respiratory mechanics CT scans were taken at PEEP levels ranging from 7.5 to 20 cmH₂O in steps of 2.5 randomly selected as either an increasing or decreasing titration. Before each PEEP level was set, the lung was fully recruited. After recruitment (with pressure control mode with PEEP=30 cmH₂O, peak inspiratory pressure=20 cmH₂O for approximately 30s), the subject was ventilated for a 10 m stabilization period with a frequency (f)=16 breaths/m, V_{T}=10 ml/kg, inspiratory to exspiratory ratio (I:E)=1:3, and F_{I}O₂=1, at the designated level of PEEP after which hemodynamic, gas exchange, and respiratory mechanics were measuresd. A whole lung CT scan was also obtained after all other measurements were collected while maintaining a constant pressure level equal to the designated PEEP. CT scans were acquired with a PQ 5000 helical single slice scanner as sold by Picker International of Cleveland, Ohio. The settings were 120 kVp, 300 mA, 8 mm thickness, with 4-mm table movement between slices, helical pitch=1.5, 0 degree tilt for scanning.

CT image analysis was performed using the Matlab software sold by Mathworks, Inc. of Natick, Mass. For each slice, lung boundaries were delineated using a semi-automatic algorithm that employed a thresholding technique and manual evaluation of the scan to ensure the accuracy of the lung boundary, adjusting the boundary as necessary. After accounting for all the Hounsfield Units (HU) voxel values within the lung regions in all slices, the mean density (HUₘₑₐₙ), standard deviation (HU_{SD}), and coefficient of variation (HU_{CV}=HU_{SD}/HUₘₑₐₙ) was calculated for all scans and the gas/tissue ratio was found for each voxel, from which total air, tissue volume, and combined (air+tissue) volume were calculated for the entire lung.

The total lung volume was divided into aeration compartments based on the HU value for each voxel (non-aerated: +100<HU<−100, under-aerated: −100<HU<−500, normally aerated: −500<HU<−800, and over-aerated: −80021 HU<−1000). A value of −800 was chosen as the threshold between the normally and over-aerated tissue compartments. This value was determined using a methodology in which healthy subject CT scans were analyzed at PEEP levels 5 and 25 cmH₂O for their HU distribution. Three dimensional lung volume meshes were created by masking lung boundary and aeration compartments. Significant alveolar recruitment was defined as a significant decrease in non-aerated lung volume with a corresponding increase in normally aerated lung volume (compared to the amounts present in each compartment at the lowest PEEP level of 7.5 cmH₂O). Alveolar overdistension was defined as a significant increase in over-aerated lung volume when compared to that obtained at the lowest PEEP level of 7.5 cmH₂O.

Regional and total static P-V curves for a single matched transverse CT slice were taken at mid-lung level. Each scan slice was divided into horizontal regions based on anatomical landmarks visible in all scans. For each region and at each PEEP level, air volume was calculated. To compare the shape of regional P-V curves within a slice, each curve was normalized by first subtracting off the air volume at the lowest PEEP level for all points and then dividing each value by the air volume at the highest PEEP level. This resulted in the regional P-V curves having a total excursion between 0 and 1 normalized air volume. Total slice P-V curves were reconstructed by summing regional air volumes at each PEEP level.

Respiratory system mechanics were monitored using an Enhanced Ventilator Waveform (EVW) with inspiratory flow profile consisting of five sinusoids ranging from 0.2 Hz to 8 Hz and expiration is passive as disclosed in U.S. Pat. No. 6,435,182, the disclosure of which is hereby incorporated by reference. A modified ventilator (the NPB840 product sold by Puritan Bennett/Tyco Healthcare of Pleasanton, Calif.) was used to deliver the EVW with the same V_{T} and fundamental frequency throughout the experiment. Airway flow was measured with a pneumotachograph (Model 4700 sold by Hans Rudolph of Kansas City, Mo.) connected to a pressure transducer (Model LCVR, 0-2 cm H₂O sold by Celesco of Chatsworth, Calif.) and trans-respiratory pressure was measured with a pressure transducer (Model LCVR, 0-50 cm H₂O product sold by Celesco of Chatsworth, Calif.) placed at the distal end of the endotracheal tube. Signals were low pass filtered at 10 Hz (4 Pole Butterworth filter product sold by Frequency Devices of Haverhill, Mass.) and sampled at 50 Hz (Modes BNC-2110 and DAQCard-6062E, National Instruments Corporation, Austin, Tex.). Inspiratory segments of flow and pressure were isolated and dynamic respiratory system resistance and elastance as a function of frequency determined.

Based on the R and E spectra, the amount of recruitment and mechanical heterogeneity during ventilation period were quantified based on two key indices: 1) E_{low} defined as E at 0.2 Hz indicative of derecruitment and respiratory tissue stiffness as well as mechanical heterogeneity and 2) Rₕₑₜ defined as the absolute difference between R at 0.2 Hz and R at 8 Hz which computational modeling studies have shown is indicative of mechanical time constant heterogeneities in the lung.

All data are presented as means±standard deviations. PEEP dependence of all variables was analyzed by one-way analysis of variance (ANOVA) for repeated measures. Between treatment differences were considered statistically significant at p<0.05. Student-Newman-Keuls analysis was used for all pair-wise comparisons.

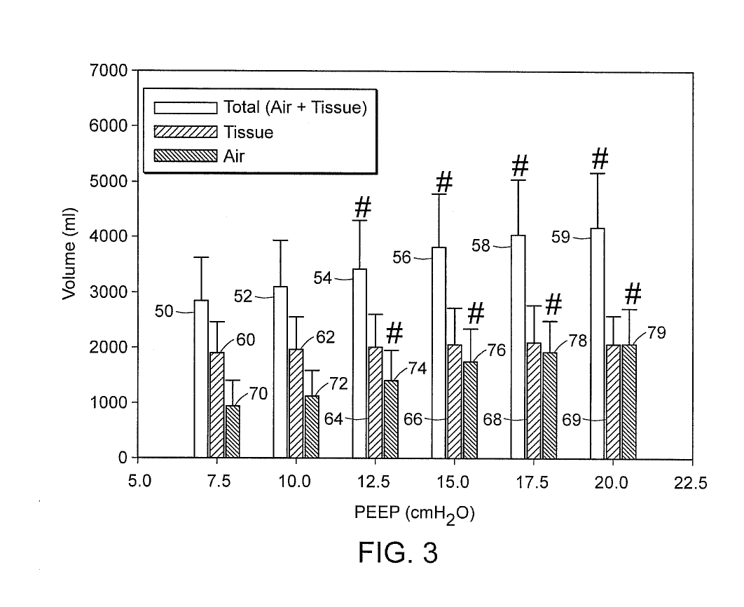

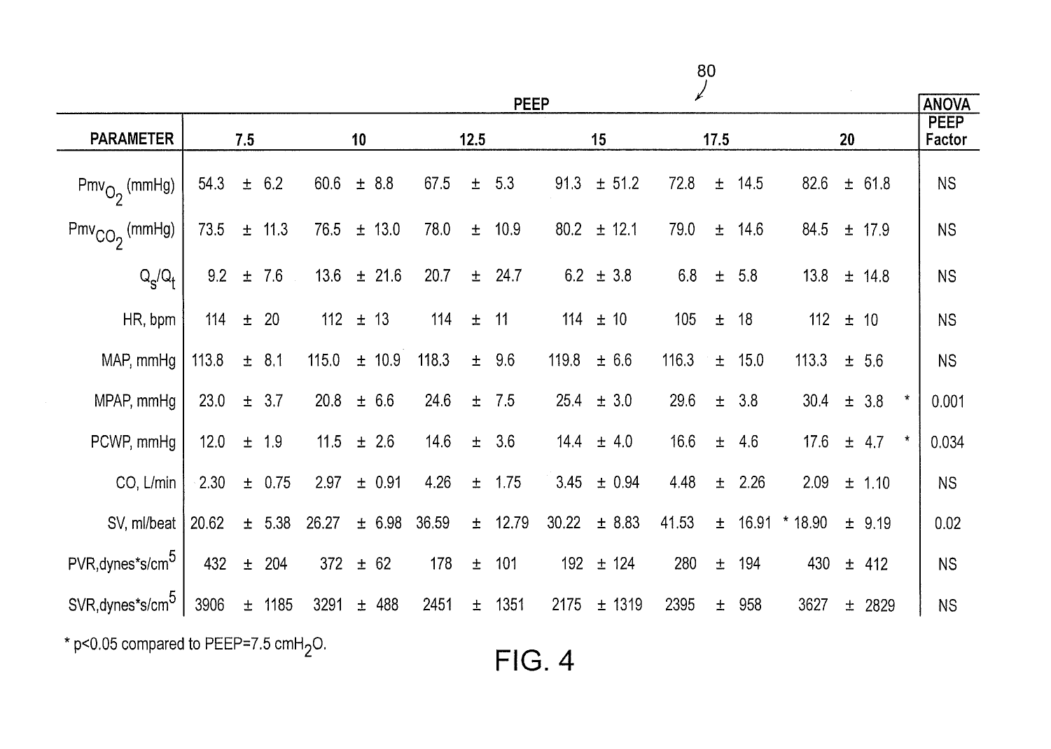

FIG. 3 shows the total volume of air and tissue in each CT scan with increasing PEEP. In particular, total (air+tissue) volume is shown at 50, 52, 54, 56, 58 and 59 for PEEP values of 7.5, 10.0, 12.5, 15.0, 17.5, and 20.0 respectively. Tissue volume is shown at 60, 62, 64, 66, 68 and 69 respectively, and air volume is shown at 70, 72, 74, 76, 78 and 79 respectively. There was a significant PEEP dependence of both the total lung volume (air+tissue) (p<0.001) and total air volume (p<0.001). The total tissue volume of the lung did not change with PEEP. The total air content in the lung increased significantly (p<0.001) from PEEP=7.5 to PEEP=12.5-20 cmH₂O. There was a significant PEEP dependence of HUₘₑₐₙ (p<0.001), HU_{SD}, HU_{COV}. Pair-wise comparisons of PEEP levels did not reveal any significant differences in HU_{SD} from PEEP=7.5 at any other PEEP level (as shown at 80 in FIG. 4). In particular, the table shown in FIG. 4 shows the analysis of variance of a variety of parameters for different PEEP values, including partial pressure of arterial carbon dioxide (Pa_{CO2}), partial pressure of mixed venous oxygen (Pmv_{CO2}), partial pressure of mixed venous carbon dioxide (Pmv_{CO2}), the shunt fraction (Qₛ/Qₜ), heart rate (HR), mean arterial pressure (MAP), mean pulmonary arterial pressure (MPAP), pulmonary capillary wedge pressure (PCWP), cardiac output (CO), stroke volume (SV), pulmonary vascular resistance (PVR), systemic vascular resistance (SVR). The valued are presented at mean +/− standard deviation. However, there were significant decreases in HUₘₑₐₙ between PEEP=7.5 cmH₂O and PEEP=12.5-20 cmH₂O (p<0.001) and significant decreases in HU_{COV} at PEEP=15-20 cmH₂O.

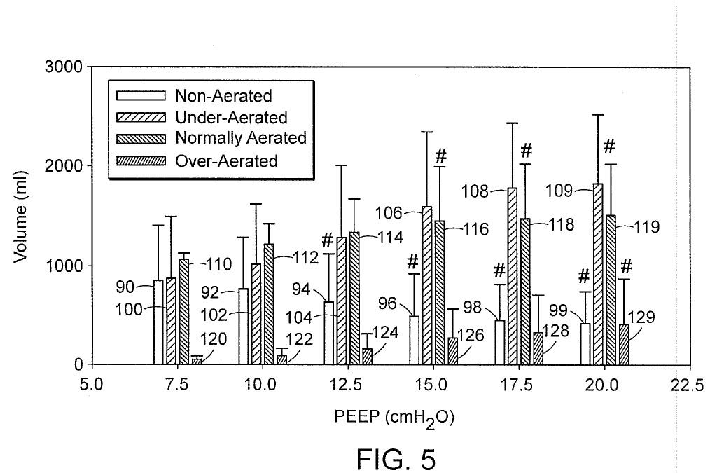

FIG. 5 shows the regional and total P-V curves reconstructed for a single matched transverse CT slice taken at mid-lung level in two representative subjects: one with a mild, diffuse distribution of disease (upper panel, subject 3) and one with a more severe, gravity-dependent distribution of disease (lower panel, subject 2). The scan at 7.5 cmH₂O for subject 2 was corrupted and not used in the analysis. Of the five subjects studied, three exhibited a diffuse distribution and two exhibited a more severe, dependent distribution. The regional P-V curves for the diffuse case nearly overlap and closely resemble the overall P-V curve with regard to shape and upper and lower inflection points. In contrast, the regional P-V curves for the localized case are dramatically different from each other in terms of their shape and inflection points, and appear to be distributed about the overall P-V curve. The overall P-V curves for the diffuse and localized cases are not distinct from each other.

FIG. 5 shows the total volume of tissue present in the different aeration compartments within the lung. In particular, FIG. 5 shows the volumes for various PEEP values at 90, 92, 94, 96, 98 and 99 for a non-aerated lung. FIG. 5 also shows the volumes for the same PEEP values at 100, 102, 104, 106, 108 and 109 for an under-aerated lung. FIG. 5 also shows the volumes for the same PEEP values at 110, 112, 114, 116, 118 and 119 for a normally aerated lung. FIG. 5 also shows the volumes for the same PEEP values at 120, 122, 124, 126, 128 and 129 for an over-aerated lung. There was a significant PEEP dependent decrease in the non-aerated lung volume (p<0.001) and significant PEEP dependent increases in the normally aerated lung volume (p<0.001) and over-aerated lung volumes; however, a significant change in the under-aerated compartment was not detected. Significant decreases in non-aerated lung volume from the initial PEEP of 7.5 cmH₂O occurred at PEEP=12.5-20 cmH₂O (p<0.001 for PEEP=15-20 cmH₂O). This corresponded to significant increases (p<0.01) in the normally aerated lung volume from the initial PEEP occurring at PEEP=15-20 cmH₂O. Therefore, increases in PEEP from 7.5 cmH₂O resulted in significant alveolar recruitment from 15-20 cmH₂O. Simultaneous and significant increases in the amount of over-aerated lung volume at PEEP=20 cmH₂O, however, were also observed.

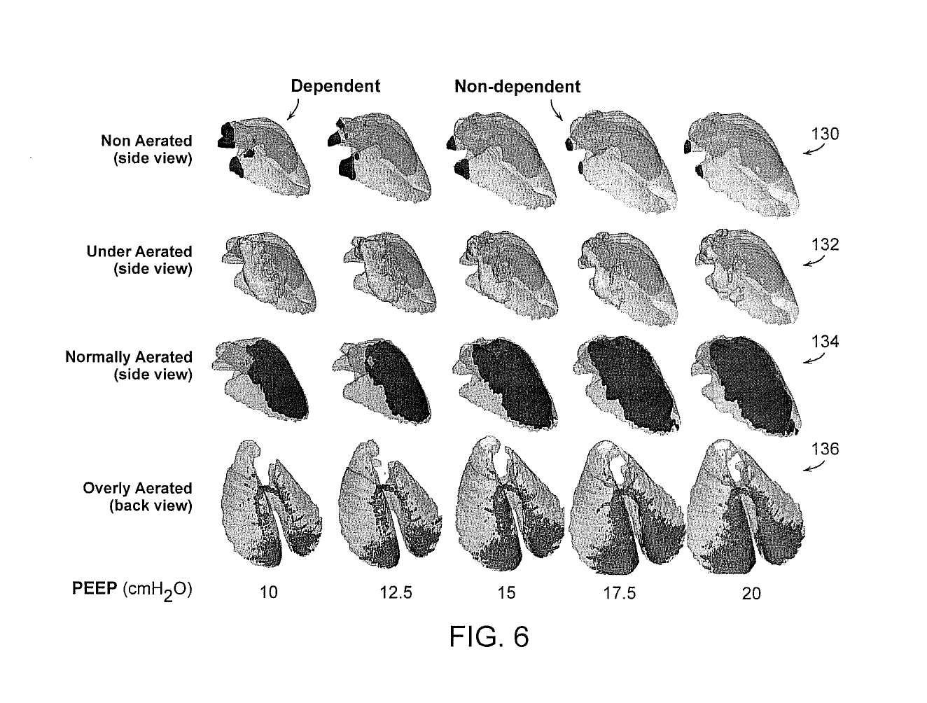

FIG. 6 shows the anatomical location of the non-aerated, under aerated, normally aerated, and over-aerated lung volumes at 130, 132, 134 and 136 respectively for a range of PEEP values in a typical prone subject with a severe, gravity dependent pattern of injury. In general, the anatomical location of the over-aerated lung was in non-dependent lung regions while recruitment occurred in more dependent regions (normally aerated volume expansion into under and non-aerated compartments).

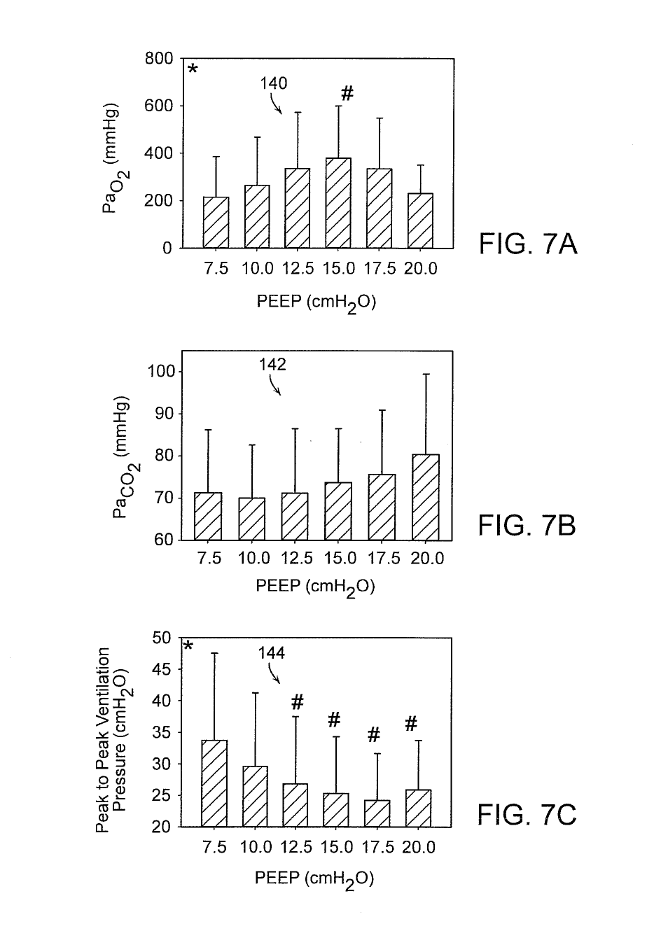

FIGS. 7A-7C show the PEEP dependence of Pa_{O2}, Pa_{CO2}, and the peak-to-peak ventilation pressures. In particular, as shown at 140 in FIG. 7A, arterial partial pressure of O₂ (Pa_{O2}) displays a significant PEEP dependence, reaching a maximum value at PEEP=15 cmH₂O and decreasing thereafter. As shown at 142 in FIG. 7B, the arterial partial pressure of CO₂ reaches a minimum at PEEP of 10.0 cm H₂O. As shown at 144 in FIG. 7C, the peak-to-peak pressures necessary to deliver ventilation reached a minimum at PEEP=17.5 cmH₂O but then showed a non-significant tendency to increase at PEEP of 20 cmH₂O. The use of the normocapnic V_{T} under control conditions resulted in considerable hypoventilation after injury, reflecting the increased dead space induced by the injury. Pa_{CO2} did not exhibit a significant PEEP dependence, however, on average this variable increased with increasing PEEP. Only MPAP, PCWP, and SV demonstrated a significant dependence on PEEP. Both MPAP and PCWP exhibited significant increases from 7.5 cmH₂O to 20 cmH₂O PEEP. SV, however, only showed a significant increase from 7.5 cmH₂O at 17.5 cmH₂O PEEP and appeared to decrease again at 20 cmH₂O.

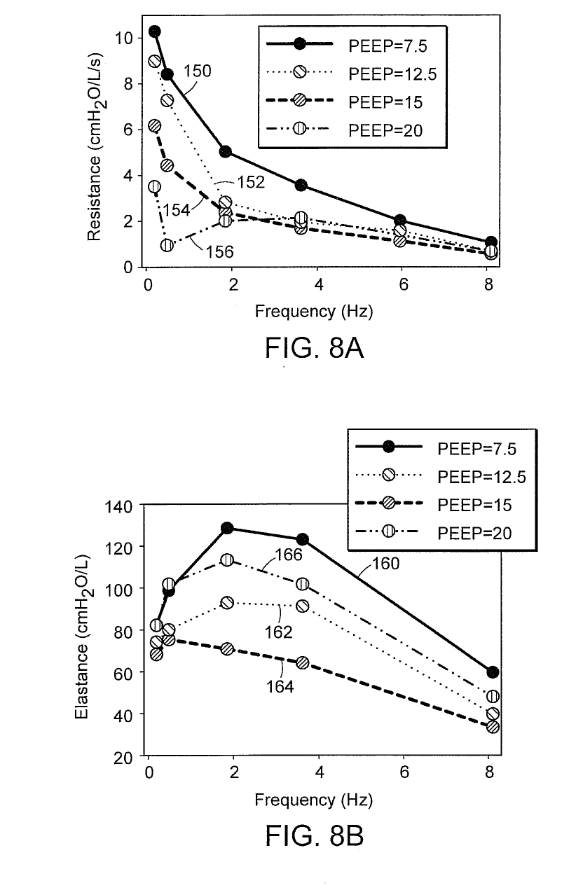

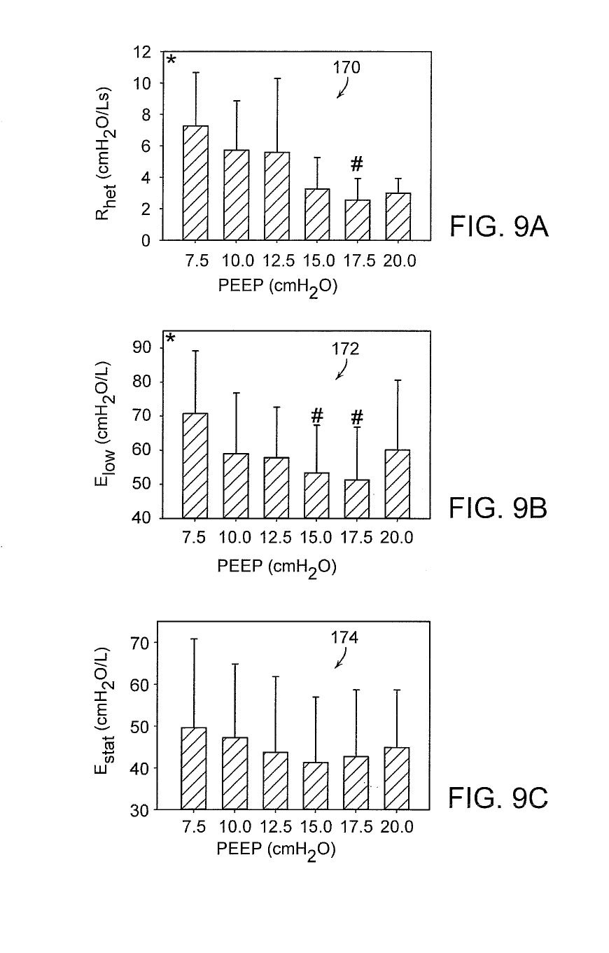

FIGS. 8A and 8B show representative R and E spectra with increasing PEEP for a typical animal (animal 3). As PEEP is increased from 7.5 cmH₂O, both the levels and frequency dependencies of R and E decreased up to 15 cmH₂O. As PEEP is further increased to 20 cmH₂O, R decreased again consistent with further dilation of the airways. However, E at 20 cmH₂O begins to increase again and become more frequency dependent. In this particular case, a PEEP of 15 cmH₂O minimizes both the levels and frequency dependence of R and E (i.e., overall mechanical heterogeneity). Both Rₕₑₜ and E_{low} displayed a significant PEEP dependence (E_{low}: p<0.01), although Eₛₜₐₜ was not significantly dependent on PEEP as shown in FIGS. 9A-9C. Pair-wise comparisons demonstrated a significant decrease in Rₕₑₜ at 17.5 cmH₂O and significant decreases in E_{low} from 15 cmH₂O (p<0.01) to 17.5 cmH₂O as compared to PEEP=7.5 cmH₂O.

The plurality of characteristic values (R and E) for each of the plurality of frequencies at each PEEP value may be generated by individually obtaining each value fo reach setting, or by employing the Enhanced Ventilator Waveform as discussed above. Once the characteristic values are identified, the relationships are analyzed to determine an optimal PEEP value.

For example, one technique employs selecting the PEEP value for which either R or E or both R and E are most linear. With reference to FIG. 8A, it may be identified that the curve for PEEP=15 (curve 154) is most linear among the set of curves 150, 152, 154 and 156. In this example, the curve for PEEP=15 is also the most linear for the set of E curves 160, 162, 164 and 166 (curve 154) as shown in FIG. 8B. The optimal PEEP value may be chosen for the PEEP value for which both R and E are most linear, may be chosen for the value for which either R or E is most linear (if not the same PEEP value), or may be chosen a value that is provided by interpolating or averaging between measured values.

A PEEP value may be chosen, for example, based only on R that is provided by an averaging or interpolation between the PEEP values of 15 and 20 (curves 154 and 156). An averaged PEEP value based on R might be (15+20)/2=17.5, while an interpolated value (for e.g., 2 Hz) might be for example ((2.25 cmH₂O/L/s×15)+(2.00 cmH₂O/L/s×20))/(2×2.125 cmH₂O/L/s)=17.35 where R=2.25 at 2 Hz for PEEP=15 and R=2.00 at 2 Hz for PEEP=20.

In further embodiments, the optimal PEEP may also be the PEEP value for which either (or both) the resistance and elastance are at a minimum for one or more of the frequencies in the range of frequencies. In further embodiments, the optimal PEEP may also be the PEEP value for which either (or both) the resistance and elastance are at a minimum average value for the range of frequencies.

It has been discovered that saline lavage in subjects results in a distribution of injury that can range from a localized or diffuse. Regardless of distribution, increases in PEEP resulted in lung recruitment as well as overdistenstion. Static measures of global lung mechanics (static P-V curve or Eₛₜₐₜ do not provide insight into the PEEP dependence of regional mechanics during ventilation. From the CT analysis, a range of PEEP values was identified over which lung recruitment was maximized without inducing significant overdistension (15 to 17.5 cmH₂O). Measures of dynamic lung mechanics (E_{low} and Rₕₑₜ) also appear sensitive to the impact of PEEP on recruitment relative to overdistension and predicted the same range of optimal PEEP. This range of PEEP also maximized oxygenation and minimized peak-to-peak ventilation pressures. There exists, therefore, an optimal PEEP during saline lavage-induced lung injury at which lung function is maximized. The above findings that measures of mechanical heterogeneity corroborate the CT quantification of disease heterogeneity, reinforces the potential of a non-invasive approach to guide ventilator settings so as to minimize the negative effects of ventilator association lung injury. Moreover, global measures of static lung mechanics are inadequate for such use since they can similar for different regional distributions of injury.

Thoracic CT allows for accurate measures of the distribution pulmonary volume and thus impact of PEEP on alveolar recruitment and overdistension during lung injury. While variations in regional static lung mechanics may exist, such variations are not evident in the total P-V curve. The CT data indicates that while PEEP increases the total air volume in the injured lung, the total tissue lung tissue was unchanged, which confirms that acute changes in pulmonary blood volume were not responsible for the functional effects of PEEP. Additionally, increases in aerated lung volume occurred heterogeneously, such that significant recruitment in dependent regions was accompanied by significant overdistension in other, predominantly non-dependent, lung regions. The above investigation was performed over the whole lung and a range of (i.e., more than two) PEEP values. By assessing this in an animal model of ARDS, a large number of entire lung CT scans were acquired at many PEEP levels. This data permitted the determination an optimal range where PEEP induced significant recruitment without significant overdistension from a baseline PEEP value.

In the above example, it was assumed that the threshold between over-aerated lung and normally aerated lung occurs at −800. The threshold employed for defining the over-aerated lung compartment was based on the HU distribution found in CT scans of the same resolution taken in healthy subject lungs at TLC. Other possible cutoffs range from about −800 to about −900, however, the absolute cutoff for this compartment could vary based on the unreliability of HU as absolute numbers, CT slice thickness, and overall voxel size relative to species lung size and anatomy. The threshold of −800 appears to provide a reliable separation between over and normally aerated lung based on the size of subjects and the resolution of CT scans used in this particular example.

A significant PEEP dependence of gas exchange (i.e., Pa_{O2}), peak-to-peak ventilation pressures, and hemodynamics (i.e., MPAP, PCWP, and SV) were found. Some of these variables increased with PEEP as expected (MPAP, PCWP). An unexpected finding was that Pa_{O2} increased with PEEP up to a level of 15 cmH₂O but further increases in PEEP decreased Pa_{O2}. Previous studies relating PEEP to gas exchange during lung injury have consistently demonstrated a positive dependence of Pa_{O2} on PEEP(11, 48). The above experiment reported a decrease in Pa_{O2} with PEEP above 15 cmH₂O. An explanation for this decrease in Pa_{O2} at the higher PEEP levels could be related to the significant increase in over-aerated lung also found at 20 cmH₂O PEEP. It has also been found that SV increased up to a PEEP of 17.5 cmH₂O, beyond which it decreased. The observed increases in PVR, PCWP, and MPAP also correspond to this mechanism. In general, the data supports that an optimal PEEP that minimizes functional heterogeneity and maximizes gas exchange also minimizes deleterious effects on pulmonary hemodynamics.

Because increases in gas volume with PEEP throughout the lung is markedly dependent on disparities in regional compliances, PEEP should be set in a way that the regional distribution of lung compliance is most homogeneous, resulting in the more homogeneous ventilation distribution with minimal risk of lung injury. While the above data demonstrate that global measures of static lung mechanics do not reflect disparities in regional lung compliance, heterogeneous lung mechanics may vary substantially, making such disparities apparent. By using global measures of static mechanics to set an optimal PEEP, one could select a value that may result in further lung injury due to the distributed nature of opening and overdistension pressures. It is important to note that while the above aeration distribution analysis was based on whole lung CT, the regional P-V curves are taken in a limited sample of the lung and may not reflect whole lung regional mechanics disparities. While CT may provide information regarding the impact of ventilator settings on regional lung mechanics, it is not a clinically practical at the bedside in critically ill patients. Moreover, under dynamic cyclic conditions, the distribution of mechanical time-constants governs ventilation distribution (not local static compliances). It has been shown that the frequency dependence of R and E embodies information related to disparities in mechanical time constants in the lung, which is directly related to the distribution of compliance. Minimizing specific dynamic mechanical indices of respiratory mechanics permits predicting levels of PEEP that maximize recruitment without inducing overdistension. If disparities in regional compliance are in fact minimized at these PEEP levels, it would also be expected that this would to lead to a more homogeneous ventilation distribution, resulting in optimal gas exchange and minimum distending pressures and this was shown in the present study.

It is important to note that the saline-lavage injury we used resulted in a highly heterogeneous distribution of injury that resulted in some level of over-aeration at all PEEP levels. In this example, an objective was to confirm that increases in PEEP can lead to both recruitment as well as overdistension and further show that dynamic mechanics indices are more sensitive to these processes than are measures of static mechanics.

Measures of dynamic respiratory mechanics are more sensitive than static measures for detecting recruitment and overdistension. The assessment of frequency dependence Rᵣₛ and Eᵣₛ in a patient may lead to an improved method for setting PEEP. The optimal range of PEEP as assessed by CT also corresponds to PEEP levels that resulted in the highest Pa_{O2} levels. Oxygenation, however, may not be the best outcome measure for patients with lung injury. The lavage model is known to be a highly recruit-able lung injury model thus making it suitable to determine the relationship between PEEP, recruitment, and dynamic respiratory mechanics. Because of this choice of subject model however, the results may be more applicable to cases of neonatal RDS. Through the assessment of dynamic lung mechanics, a PEEP level may be selected that balances the tradeoff between recruitment and overdistension as assessed via CT.

The results support the notion that an optimal PEEP exists that maximizes lung function during lung injury (i.e., recruitment relative to overdistension, Pa_{O2}, peak-to-peak ventilation pressures, and mechanical heterogeneities), which may not be apparent in measures of static lung mechanics alone. Depending on the physical distribution of injury, this optimal PEEP may not be evident in global measures of static lung mechanics. The assessment of dynamic respiratory mechanics could be used to set PEEP to minimize the distribution of disease.

Those skilled in the art will appreciate that numerous modifications and variations may be made to the above disclosed embodiments without departing from the spirit and scope of the invention.

What is claimed is:

1. A method for determining a peak end expiratory pressure in a mechanical ventilator system for providing respiratory assistance to a patient, said method comprising the steps of:
determining characteristic values for a plurality of frequencies at each of a plurality of peak end expiratory pressures; and
selecting an optimal peak end expiratory pressure value responsive to said characteristic values.

2. The method as claimed in claim 1, wherein said optimal peak end expiratory pressure value is the peak end expiratoly pressure value for which said characteristic values are most linear with said plurality of frequencies.

3. The method as claimed in claim 1, wherein said optimal peak end expiratory pressure value is interpolated from peak end expiratory pressure values for which said characteristic values are determined.

4. The method as claimed in claim 1, wherein said optimal peak end expiratory pressure value is averaged between peak end expiratory pressure values for which said characteristic values are determined.

5. The method as claimed in claim 1, wherein said characteristic values include resistance.

6. The method as claimed in claim 1, wherein said characteristic values include elastance.

7. The method as claimed in claim 1, wherein said characteristic values include resistance and elastance.

8. The method as claimed in claim 1, wherein said plurality of frequencies includes at least four.

9. The method as claimed in claim 1, wherein said respiratory assistance is provided to patient suffering from acute respiratory distress syndrome.

10. The method as claimed in claim 1, wherein said respiratory assistance is provided to patient suffering from an acute lung injury.

11. A method for determining a peak end expiratory pressure in a mechanical ventilator system for providing respiratory assistance to a patient, said method comprising the steps of:
determining characteristic values for a plurality of frequencies at each of a plurality of peak end expiratory pressures;
identifying an identified peak end expiratory pressure value for which said characteristic values are most linear for said plurality of frequencies; and
selecting an optimal peak end expiratory pressure value responsive to said identified peak end expiratory pressure value.

12. The method as claimed in claim 11, wherein said optimal peak end expiratory pressure value is the identified peak end expiratory value.

13. The method as claimed in claim 11, wherein said optimal peak end expiratory pressure value is interpolated from peak end expiratory pressure values for which said characteristic values are determined.

14. The method as claimed in claim 11, wherein said optimal peak end expiratoly pressure value is averaged between peak end expiratory pressure values for which said characteristic values are determined.

15. The method as claimed in claim 11, wherein said characteristic values include resistance.

16. The method as claimed in claim 11, wherein said characteristic values include elastance.

17. The method as claimed in claim 11, wherein said characteristic values include resistance and elastance.

18. The method as claimed in claim 11, wherein said respiratory assistance is provided to patient suffering from acute respiratory distress syndrome.

19. The method as claimed in claim 11, wherein said respiratory assistance is provided to patient suffering from an acute lung injury.

20. The method as claimed in claim 11, wherein said identified peak end expiratory pressure value is a peak end expiratory pressure value for which said characteristic values are at a minimum for a frequency within said plurality of frequencies.

21. A system for determining a peak end expiratory pressure in a mechanical ventilator system for providing respiratory assistance to a patient, said system comprising:
collection means for determining characteristic values for a plurality of frequencies at each of a plurality of peak end expiratory pressures;
identification means for identifying an identified peak end expiratory pressure value for which said characteristic values are most linear for said plurality of frequencies; and
selection means for selecting an optimal peak end expiratory pressure value responsive to said identified peak end expiratory pressure value.
